(19) United States
(12) Patent Application Publication
(10) Pub. No.: US 2020/0254214 A1
(43) Pub. Date: Aug. 13, 2020

(54) PORTAL VEIN ACCESS DEVICE AND METHOD FOR FACILITATING PORTAL VENOUS ENTRY FOR CREATION OF A PERCUTANEOUS TRANSJUGULAR INTRAHEPATIC PORTOSYSTEMIC SHUNT

(71) Applicant: Timothy Patrick Murphy, Providence, RI (US)

(72) Inventor: Timothy Patrick Murphy, Providence, RI (US)

(21) Appl. No.: 16/273,462

(22) Filed: Feb. 12, 2019

(51) Int. Cl. A61M 25/00; A61M 25/09; A61B 17/34

(52) U.S. Cl. CPC A61M 25/0026; A61M 25/0662; A61B 17/3403; A61M 25/09041

(57) ABSTRACT

The invention features is an embodiment of methods and devices that comprise a multilumen portal vein access device, comprising at least a lumen for passage of a needle or needle-tipped guide wire that is used for piercing the liver from a hepatic vein position to a branch of the portal vein through the substance of the liver, and at least one other lumen that is use for passage of a guide wire, said other lumen and guide wire serving the function of preserving the position of the portal vein access device within the hepatic vein, thereby facilitating multiple attempts at puncturing the portal vein without the need to select the hepatic vein with separate maneuvers between needle advancement attempts.

This application claims priority under 35 U.S.C. § 119 and 35 U.S.C. § 120 of provisional patent application No. 62/652,392, EFS ID 32241511, filed Apr. 4, 2018, entitled “Method for Facilitating Portal Venous Entry for Creation of a Percutaneous Transjugular Intrahepatic Portosystemic Shunt (TIPS) and Portal Vein Access Device”, by inventor Timothy Murphy, attorney docket number MUR-20180403, the entirety of which is incorporated herein by reference in its entirety.

### REFERENCES

Rossle M. TIPS: 25 years later. Journal of Hepatology 2013 vol. 59:1081-1093

U.S. Pat. No. 7,722,567B2

U.S. Pat. No. 8,287,481B1

U.S. Pat. No. 8,628,491B2

U.S. Pat. No. 8,632,468B2

U.S. Pat. No. 9,072,596B1

US20100217117A1

US20130245533A1

U.S. Pat. No. 5,328,480A

U.S. Pat. No. 5,354,279A

U.S. Pat. No. 5,464,395A

U.S. Pat. No. 8,374,680B2

US20050222581A1

US20140180098A1

US20140343482A1

US20160015422A1

US20160331941A1

### FIELD

The present invention relates to methods and devices for performing surgical procedures, and in particular to methods and devices for performing a shunt procedure between branches of the portal vein and branches of the hepatic vein in the liver.

### BACKGROUND OF THE INVENTION

Portal hypertension is a medical condition characterized by high blood pressure in the mesenteric or visceral veins in the abdomen, including the portal vein, visceral veins, and their tributaries. Portal hypertension is most often caused by disease of the liver, usually cirrhosis of the liver, but can be caused by any disease that increases flow into the portal circulation, such as for example arteriovenous communication, malformation, or fistula between the arterial circulation and the portal vein or its tributaries, or by restriction of flow of the portal vein caudal to the liver (“pre-hepatic”), within the liver (“hepatic”) or cephalad to the liver (“post-hepatic”). Portal hypertension results in several medical complications that can be severe and life-threatening, including hemorrhagic complications such as upper gastrointestinal hemorrhage. Upper gastrointestinal hemorrhage associated with portal hypertension is usually caused by dilated veins, called “varices” that try to bypass the diseased liver, but can also be caused for example by portal gastropathy or portal gastropathy caused by portal hypertension.

Most commonly the dilated veins originate from the portal or splenic veins, and ascend through the abdomen as coronary or gastric varices and then often into the chest as esophageal varices. Less commonly, varices can be isolated in the stomach, draining in the retroperitoneum through a spontaneous venous connection to a renal vein, which drains into the vena cava and right atrium without having to go through the diseased liver or esophageal veins. In addition to hemorrhage, elevated pressures in the visceral or mesenteric veins can lead to another morbidity of portal hypertension, namely excess accumulation of fluid within the abdomen (“ascites”), or when this fluid can get access to the chest cavity through defects in the diaphragm, pleural effusions or fluid around the lungs (“hydrothorax”).

One way to treat the medical complications of portal hypertension is to create a shunt or bypass within the liver substance that allows intestinal blood to bypass the liver parenchyma and flow through the shunt directly into the draining liver veins and into the right atrium. Open surgical methods to place such a bypass are associated with much higher morbidity than percutaneous or “through the skin” methods that are more commonly used. The percutaneous method of creating a liver shunt is called “transjugular intrahepatic portosystemic shunt”, or TIPS.

The transjugular intrahepatic portosystemic shunt (TIPS) was first formed by Dr. Joseph Rosch in 1968 in a dog model. After introduction of metal stents, Dr. Julio Palmaz used stents to improve the TIPS procedure in 1985, and Dr. Goetz Richter performed the first TIPS in a human in 1988. Since then TIPS has become widespread commonly shunts are performed people with portal hypertension and upper gastrointestinal hemorrhage due to esophageal varices, gastric varices, portal gastropathy, refractory ascites or pleural effusion.

An exemplary conventional method to perform the TIPS procedure, done percutaneously with fluoroscopy or other medical imaging guidance, is as follows: access is gained usually into a jugular vein often using Seldinger's method, using a needle and guide wire, then a vascular access sheath is placed into the jugular vein, then through the vascular access sheath a second, longer sheath is introduced over the guide wire and through the vascular access sheath. The second longer sheath is often pre-loaded with a shaped catheter, or once placed a shaped catheter can be placed therethrough. The second longer sheath, catheter and guide wire are then negotiated through the jugular and brachiocephalic veins, superior vena cava, right atrium, intrahepatic inferior vena cava (IVC), and into a hepatic vein, usually the right hepatic vein. Once access is gained in the hepatic vein, said second longer sheath is advanced distally into the hepatic vein. After removal of the catheter, a second, curved needle is then passed over the guide wire through the second longer sheath, and into the hepatic vein several centimeters caudal to its confluence with the inferior vena cava. The guide wire is then removed, and the needle is unsheathed by retraction of the second larger sheath such that said needle's tip is exposed. The method then used to achieve transhepatic access of the portal vein from the hepatic vein involves a “retract —advance-retract” maneuver of the second, curved needle. That is, for example, the second, curved needle and second longer sheath are first retracted in unison with the tip of the second, curved needle located within the hepatic vein, and then torqued or rotated, often counterclockwise when in the right hepatic vein, to give an appropriate approach angle on the liver parenchyma, thereby directing the curved needle tip toward the porta hepatis and portal vein branches, facilitating access into a portal vein branch. Then, the second, curved needle is advanced through the liver parenchyma in the direction of portal vein branches. After advancing the second, curved needle into the liver substance in the direction of portal vein branches, the second, curved needle is retracted through the liver parenchyma proximally with a syringe usually connected to the needle and the needle aspirated until blood return is achieved, completing the “retract-advance-retract” maneuver for transhepatic portal vein access. Once blood return from the second, curved needle is achieved, radiopaque contrast can be injected to confirm portal vein branch access and then a guide wire advanced through the second, curved needle, hopefully passing into the portal vein and then into usually the splenic or superior mesenteric veins. Then, the second longer sheath is advanced over the second, curved needle through the parenchyma or tissue of the liver and into the portal vein branch. Next, the second, curved needle is removed and usually an angioplasty balloon advanced over the guide wire into the portal vein. This balloon is used to dilate the liver parenchymal tract, with appropriate adjustments in position of the second longer sheath. The balloon then is removed and the outer sheath is again advanced through the tract, and a tube conduit typically introduced over the guide wire into the liver parenchymal tract. The tube conduit is deployed by unsheathing or by retracting the larger outer sheath, and left in the hepatic parenchymal tract. Typically, the tube conduit is left in the liver parenchyma to preserve the portal-vein-to-hepatic vein liver parenchymal tract. Such a tube conduit could be composed of a wire mesh, a textile fabric, xenograft or allograft blood vessel, or combination thereof. This tube conduit comprises the shunt, and functions to connect the portal veins inferior to the liver with the hepatic veins, vena cava, and right atrium superior to the liver, allowing much of the mesenteric blood within the abdomen to bypass the liver parenchyma. Reduced congestion in the mesenteric veins usually relieves the varices, and ascites or pleural effusion.

A person having ordinary skill in the art will readily recognize common variations of this procedure description. For example, in some cases the procedure may be done without the first vascular access sheath, using only the longer access sheath. Also, there may be common differences in the sequence of dilation of the hepatic parenchymal tract, such as dilating the hepatic parenchymal tract before tube conduit placement, dilating the hepatic parenchymal tract after tube conduit placement, or dilating the hepatic parenchymal tract both before and after placement of the tube conduit.

The TIPS procedure is technically challenging to perform. The rate-limiting or most difficult step in the TIPS procedure is the “retract-and-advance” maneuver of the second, curved needle, by which the said second, curved needle gains access into a portal vein branch. Often the “retract-advance-retract” maneuver of the second, curved needle does not result in access into a portal vein branch and therefore multiple “retract-advance-retract” maneuvers are often required. Because of the anatomic location of the portal vein relative to the hepatic vein, usually the needle must be retracted to a central location in the hepatic vein from its more peripheral position as it entered the vein before it is advanced into the liver parenchyma in search of a portal vein. Often, the acceptable location for advancement of the needle is within a few centimeters or less of the confluence of the hepatic vein and the inferior vena cava. Adding to the difficulty, the intrahepatic inferior vena cava superior to the hepatic vein may be quite short, e.g., <3 cm, which means that when the needle prolapses from the hepatic vein into the IVC during retraction its tip often ends up positioned close to or even within the right atrium. It is recognized that the needle cannot safely be advanced from the intrahepatic IVC or from the right atrium because it is technically unlikely to enter a portal vein from this location, but even worse could cause fatal complications such as hemorrhage within the abdomen, or hemopericardium. The timing of said “retract-advance-retract” maneuver is critical to the success of the procedure, as often patient breathing results in downward excursion of the diaphragm, and thereby the liver, on inhalation, resulting in prolapse of the second, curved needle into the IVC or right atrium. There are two opportunities for retraction of the portal vein access device to prolapse out of the hepatic vein and into the IVC or right atrium, first during the initial retraction prior to transhepatic access, and second after transhepatic access during retraction of the needle in an effort to return portal venous blood. Thus, retraction of the second, curved needle within the hepatic vein is precarious.

When a “retract-advance-retract” maneuver of the second, curved needle is done and portal vein access is not achieved, and the second, curved needle and second longer sheath are too proximal to advance again safely without repositioning in the hepatic vein, prior to advancing the portal vein access device a guide wire will need to be reintroduced through the needle. If the needle is still in position within a hepatic vein the needle and second longer sheath may advance over the needle and another “retract-advance-retract” attempt at needle access into the portal vein done. However, frequently the guide wire may not be in the hepatic vein when passed down the needle after needle retraction because the second, curved needle is too central and in or close to the IVC, and if so the needle has to be removed and the catheter replaced through the second longer sheath over the guide wire so that the hepatic vein can be selected again, and then the catheter removed and the needle replaced over the guide wire, as with the preceding attempt at portal vein access. This is cumbersome, time-consuming, and potentially risky. Since it is not uncommon for TIPS procedures to require a dozen or more “retract-advance-retract” maneuvers of the second, curved needle into the liver parenchyma in an effort to gain access into the portal vein, the lack of stability or purchase of the needle tip within the hepatic vein after retraction of the needle is a major barrier to efficient performance of the TIPS procedure.

### SUMMARY

The methods and devices disclosed herein generally optimize the performance of transjugular intrahepatic portosystemic (TIPS) shunts because they maintain or preserve access of the needle and second longer sheath within the hepatic vein during “retract-advance-retract” maneuvers of the second, curved needle, thereby allowing multiple attempts at needle access of the portal vein from the hepatic vein without the need to recatheterize the hepatic vein after failed attempts, thereby reducing or eliminating exchanges of catheters, needles, and guide wires, thereby facilitating the critical step of the TIPS procedure, access into portal vein branches from a hepatic vein or vena cava.

An exemplary method and device to address the problem of loss of hepatic vein access during retraction of the conventional TIPS portal vein access needle after errant puncture disclosed herein is as follows, consisting of the following steps, not necessarily in order, usually performed using fluoroscopy or other medical imaging guidance:
access of the jugular vein using a first needle (not shown) and a first guide wire (not shown);
placement of a vascular access sheath (not shown) into the jugular vein;
placement of a multilumen portal vein access device 101 comprising at least a first lumen 11 for guide wire 13 and catheter (not shown) placement and at least a second lumen 10 for second curved needle 12 placement therethrough, said multilumen portal vein access device advanced over said guidewire and through said vascular access sheath;
placement of a catheter (not shown) over said guide wire through a first lumen of said multilumen portal vein access device 101 to traverse the heart and selectively entering one of the following veins: left hepatic vein, middle hepatic vein, right hepatic vein 26, common hepatic vein;
advancement of said guide wire and catheter distally into said vein;
advancement of said portal vein access device 101 over said guide wire and said catheter into said vein such that said portal vein access device needle exit port 17 resides at least 3 cm from the confluence of said vein with the inferior vena cava while catheter or guide wire purchase is maintained in the more peripheral hepatic vein, for example, in the hepatic vein for a distance of between 5 cm and 30 cm;
introduction of said second, curved needle 12 into said second lumen of said portal vein access device so that it passes through said second lumen to but not out of said needle exit port 17;
adjustment of position of said portal vein access device 101 in said vein so that advancement of said second, curved needle 12 out of said needle exit port is anatomically likely to enter a portal vein branch;
advancement of said second, curved needle 12 through said second lumen of into said liver parenchyma and into a portal vein or portal vein branch 27;
advancement of a second guide wire (not shown) through said second, curved needle 12 into the portal vein;
removal of said portal vein access device 101 and said first guide wire 13;
removal of second, curved needle 12 over said second guide wire;
dilation of the parenchymal tract using an angioplasty balloon or dilator (not shown) introduced over said second guide wire;
deployment of a tube conduit (not shown) to preserve the portal-vein-to-hepatic vein liver parenchymal tract over said second guide wire;
removal of said tube conduit introducer, said second guide wire, and said large-bore vascular access sheath.

It can be readily appreciated that there a multiple means by which purchase within a hepatic vein can be preserved despite retraction of the portal vein access device. For example, the first guide wire 13 can be used by itself to maintain purchase. An extended tip 20 of said portal vein access device beyond its needle exit port could also be used to maintain purchase. Also, a transformable mechanical modification of the tip end of said portal vein access device, such as by the addition of an expansile element, such as for example an inflatable balloon, Malecot tip, or other anchoring device, at its tip beyond the needle exit port could also help to preserve access and purchase in the hepatic vein during the needle “retract-advance-retract” maneuver.

### BRIEF DESCRIPTION OF THE DRAWINGS

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

FIG. 1 is an illustration one exemplary embodiment of the invention, which comprises a portal vein access apparatus, as seen here in longitudinal cut-away section.

FIG. 2 is an illustration of another exemplary embodiment of the invention, comprising a portal vein access apparatus, also seen in longitudinal cut-away section

FIG. 3 is an illustration of an exemplary embodiment of the invention, comprising a portal vein access apparatus, in longitudinal cut-away view.

FIG. 4 is an illustration of a cross-section of the proximal section of the embodiment seen in FIG. 2.

FIG. 5 is a schematic of the anatomy of the liver, portal veins, hepatic veins, vena cava, and right atrium.

### DETAILED DESCRIPTION OF THE DRAWINGS

Referring to the drawing figures, like reference numerals designate identical or corresponding elements throughout the several figures. The singular forms “a,” “an,” and “the” include plural referents unless the context clearly dictates otherwise. Thus, for example, reference to “a solvent” includes reference to one or more of such solvents, and reference to “the dispersant” includes reference to one or more of such dispersants.

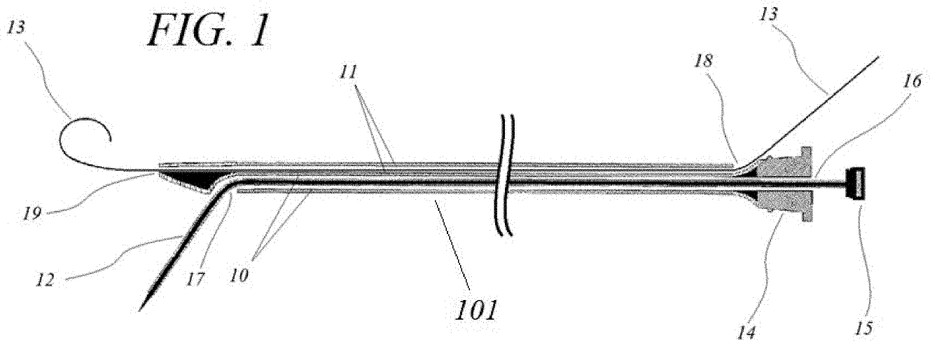

According to one embodiment of the device, as shown in FIG. 1, the portal vein access device 101 comprises a sheath with multiple lumens, in FIG. 1 two lumens are shown by way of example, one lumen 10 which accommodates a needle or needle-sheath combination 12, said needle which has a cap or handle at its hub end 15, said lumen 10 with an entry port 16 at the hub end 14 and a needle exit port 17 at its distal end, in this embodiment. The second lumen 11 accommodates a guide wire 13 with an entry port 18 at the hub end of the apparatus and a guide wire exit port 19 at the tip end.

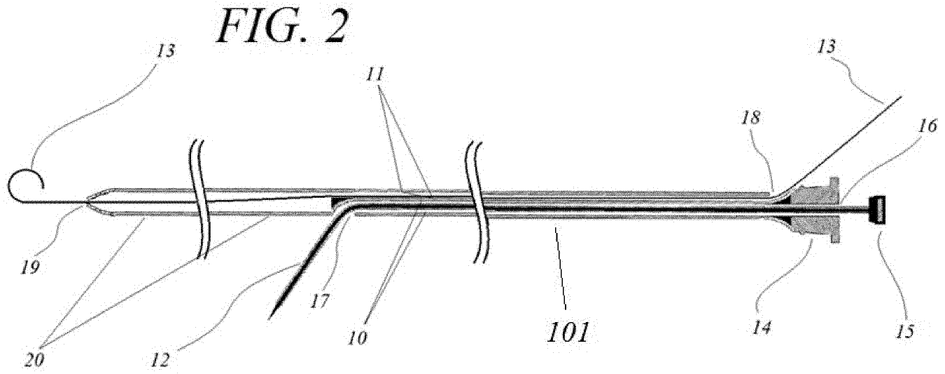

According to another embodiment of the invention, as shown in FIG. 2, the portal vein access device 101 comprises a sheath with multiple lumens, in FIG. 2 two lumens are shown by way of example, but the shaft between the needle exit port 17 and the guide wire exit port 19 of the portal vein access device 101 is elongated, and in this embodiment comprises an extended single-lumen component 20 at the distal end at the guide wire exit port 19. In this embodiment, the extended tip is of similar outer diameter to the sheath proximal to needle or needle-tip guide wire exit port, so the portal vein access device is of nearly uniform outer diameter throughout its length.

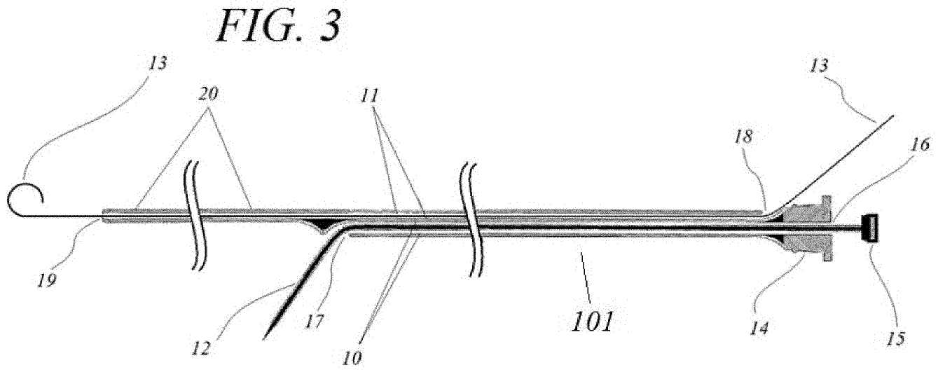

According to another embodiment of the invention, as shown in FIG. 3, the portal vein access device 101 comprises a sheath with multiple lumens, in FIG. 2 two lumens are shown by way of example, but the shaft between the needle exit port 17 and the guide wire exit port 19 of the portal vein access device 101 is elongated, and in this embodiment comprises and extended single-lumen component 20 at the tip end near the guide wire exit port 19. In this embodiment, the outer diameter of the portal vein access device is not uniform throughout its length, but rather it tapers distal to the exit port of the needle or needle-tipped guide wire, so that the distal segment without the needle or needle-tipped guide wire lumen is of narrower outer diameter than the proximal segment that includes the needle or needle-tipped guide wire lumen.

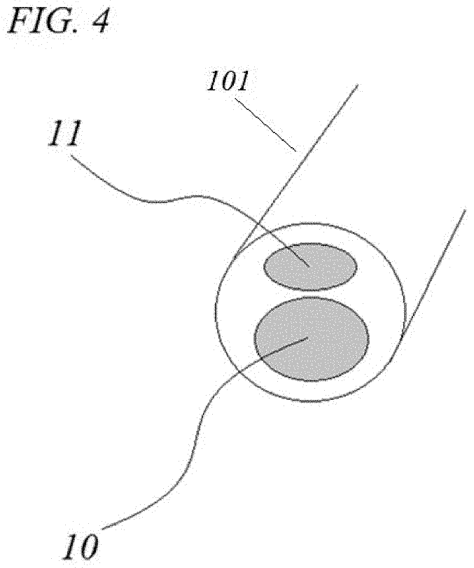

FIG. 4 is an example of an embodiment of the multi-lumen portal vein access device 101 showing a proximal segment of the apparatus in cross-section, which comprises in this example two lumens, one that accommodates the portal vein access needle or needle-cannula 10 and a second lumen 11 that accommodates a guide wire.

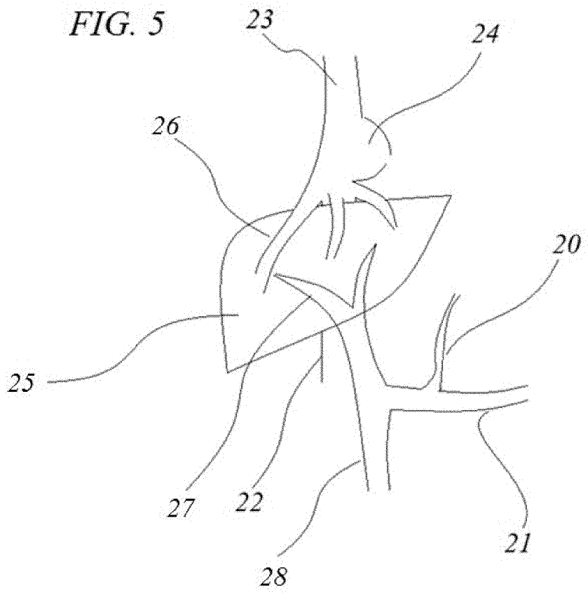

FIG. 5 is a schematic in coronal section of the liver 25, right hepatic vein 26, superior vena cava 23, right atrium 24, right portal vein 27, inferior vena cava 22, superior mesenteric vein 28, splenic vein 21, and coronary vein varix 20.

### INCORPORATION BY REFERENCE

References and citations to other documents, such as patents, patent applications, provisional patent applications, patent publications, journals, books, papers, web content, that have been made throughout this disclosure are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A portal vein access device comprising a tube with a proximal or hub end and a distal or tip end, said device containing a plurality of lumens essentially parallel to its long axis, said lumens being non-concentric, in which a first lumen accommodates a curved needle when passed from proximal to distal within said first lumen, said first lumen having a proximal end at the hub end of the portal vein access device and a distal end, said first lumen distal end occurring at a needle exit port, and also having at least one second lumen, said second lumen accommodating a guide wire through its entire length.

2. A portal vein access device of claim 1 with a first lumen needle exit port that deflects the path of the exiting needle or needle-tipped guide wire at least 5 degrees off of the long axis of said portal vein access device.

3. A portal vein access device comprising a tube with a proximal or hub end and a distal or tip end, said device containing a plurality of lumens essentially parallel to its long axis, said lumens being non-concentric, in which a first lumen accommodates a curved needle when passed from proximal to distal within said first lumen, said first lumen having a proximal end at the hub end of the portal vein access device and a distal end, said first lumen distal end occurring at a needle exit port, and also having at least one second lumen, said second lumen accommodating a guide wire through its entire length, and said second lumen having an elongated tip that it extends between 5 and 30 cm beyond the said needle exit port of said first lumen.

4. A portal vein access device of claim 3 with a first lumen needle exit port that deflects the path of the exiting needle or needle-tipped guide wire at least 5 degrees off of the long axis of said portal vein access device.

5. A method for performing a percutaneous transjugular intrahepatic portosystemic shunt procedure, using fluoroscopic or other medical imaging guidance, that includes preserving access of a guide wire to a hepatic vein during maneuvers of a needle used to access the portal vein, consisting of the following steps, not necessarily in order:
access of the jugular vein using a first needle and a first guide wire;
placement of a vascular access sheath into the jugular vein;
placement of a multilumen portal vein access device comprising at least a first lumen for guide wire and catheter placement and at least a second lumen for second curved needle placement therethrough, said multilumen portal vein access device advanced over said guidewire and through said vascular access sheath;
placement of a catheter over said guide wire through a first lumen of said multilumen portal vein access device to traverse the heart and selectively entering one of the following veins: left hepatic vein, middle hepatic vein, right hepatic vein, common hepatic vein;
advancement of said guide wire and catheter distally into said vein;
advancement of said portal vein access device over said guide wire and said catheter into said vein such that said portal vein access device needle exit port resides at least 3 cm caudal to the confluence of said vein with the inferior vena cava;
introduction of said second, curved needle into said second lumen of said portal vein access device so that it passes through said second lumen to, but not out of, said needle exit port;
adjustment of position of said portal vein access device in said vein so that advancement of said second, curved needle out of said needle exit port is anatomically likely to enter a portal vein branch;
advancement of said second, curved needle through said second lumen into said liver parenchyma and into a portal vein or portal vein branch;
advancement of a second guide wire through said second, curved needle into the portal vein;
removal of said portal vein access device and said first guide wire;
dilation of the parenchymal tract using an angioplasty balloon or dilator introduced over said second guide wire;
deployment of a tube conduit in the portal-vein-to-hepatic vein liver parenchymal tract over said second guide wire;
removal of said tube conduit stent or stent-graft introducer, said second guide wire, and said large-bore vascular access sheath.

6. A method of performing percutaneous transjugular intrahepatic portosystemic shunt procedures of claim 5, in which said tube conduit stent or stent-graft is dilated using a balloon catheter or dilator after deployment in said portal-vein-to-hepatic vein liver parenchymal tract.

7. A method of performing percutaneous transjugular intrahepatic portosystemic shunt procedures of claim 5 that includes preserving access to said hepatic vein during retraction of the portal vein access device prior to needle advancement through the hepatic parenchyma by use of an elongated tip of said portal vein access device that extends distally between 5 cm and 30 cm beyond the needle exit port.

8. A method of performing percutaneous transjugular intrahepatic portosystemic shunt procedures of claim 5 that includes preserving access to a hepatic vein during retraction of the portal vein access device prior to needle advancement through the hepatic parenchyma by use of a guide wire that extends beyond the tip of said portal vein access device, to a distance in the vein between5 cm and 30 cm beyond the needle exit port.

9. A method of performing percutaneous transjugular intrahepatic portosystemic shunt procedures of claim 5 that includes preserving access to a hepatic vein during retraction of the portal vein access device prior to needle advancement through the hepatic parenchyma by use of an expansile element on the portal vein access device located distally enough to be within the hepatic vein during retraction maneuvers of the portal vein access device during attempts at portal vein access.

10. A method of performing percutaneous transjugular intrahepatic portosystemic shunt procedures of claim 9 where said expansile element is an angioplasty balloon.

11. A method of performing percutaneous transjugular intrahepatic portosystemic shunt procedures of claim 9 where said expansile element is Malecot tip.

12. A method of performing percutaneous transjugular intrahepatic portosystemic shunt procedures of claim 9 where said expansile element is an anchoring device.
